(19) United States
(12) Patent Application Publication
(10) Pub. No.: US 2012/0199121 A1
(43) Pub. Date: Aug. 9, 2012

(54) INHALERS

(75) Inventors: Rachel Striebig, Wanstead (GB); Peter Hodson, Loughborough (GB); David Harris, Milton (GB); David Greenleaf, Loughborough (GB); Marek Myszko, Great Cambourne (GB); Amanda Jane Fuller, Coton (GB); Matthew Allen, Wanstead (GB)

(73) Assignee: CAMBRIDGE CONSULTANTS LIMITED, Cambridge (GB)

(21) Appl. No.: 13/392,144

(22) PCT Filed: Aug. 24, 2010
(86) PCT No.: PCT/GB2010/001598

§ 371 Date: Apr. 24, 2012

(30) Foreign Application Priority Data
Aug. 24, 2009 (GB) 0914738.0

(51) Int. Cl. A61M 15/00

(52) U.S. Cl. 128/203.15

(57) ABSTRACT

A dry powder inhaler incorporates a cyclone chamber (4) comprising at least one air inlet (6) and an outlet (10) wherein the configuration of the air inlet (6) and the shape of the chamber (4) is such that in use a reverse flow cyclone is set up in the chamber (4). The chamber (4) further comprises flow disrupter means, e.g. the upper surface (14) of a protrusion (12) from the base, disposed so as in use to restrict air flowing from the base of the chamber (4) in an axial direction.

This invention relates to dry powder inhalers. It relates particularly, although not exclusively, to dry powder inhalers employing a reverse cyclone, as taught for example in our earlier WO 2006/061637. In the system disclosed therein, a powder formulation of micronized drug particles mixed with coarser, non-respirable, lactose carrier particles is typically used, the shear forces in the outer, downwardly spiralling “free” vortex and the inner upwardly spiralling “forced” vortex (and, probably more particularly, the cyclone chamber wall collisions) serving to deagglomerate the particles (i.e. in particular to remove the respirable drug particles from the non-respirable carrier particles).

It is desirable in dry powder inhalers to minimise the size of the cyclone chamber, for example in producing an inhaler with multiple doses, each with its own cyclone chamber, as is fore-shadowed in WO 2006/061637, the maximum dimensions of each chamber will determine how many doses can be provided in an acceptably sized device. The inventions disclosed in WO 2006/061637 enable small, efficient inhalers to be produced. However the Applicant has now appreciated that there is a further constraint on reducing the size of the cyclone chamber therein.

If the cyclone chamber becomes too small, particularly when the powder with which it is initially filled takes up an appreciable proportion of the volume of the chamber, there is a tendency, particularly when inhalation first commences, for the powder to be dragged rapidly out of the chamber before the cyclone has been set up. Even after the cyclone has been set up, in such small cyclone chambers there is a tendency for the powder to be pushed to the very centre of the cyclone where it is subjected to high axial velocities without experiencing the shear forces in the vortex field (i.e. where it does not tend to be flung outwards from the central forced vortex region back into the free vortex region that surrounds it). This leads to an undesirable quantity of medicament particles leaving the chamber still attached to lactose carrier particles, and therefore too large to be respirable.

However, the Applicant has devised certain approaches for reducing the constraints set out above and when viewed from a first aspect the present invention provides a dry powder inhaler incorporating a cyclone chamber comprising at least one air inlet and an outlet wherein the configuration of the air inlet and the shape of the chamber is such that in use a reverse flow cyclone is set up in the chamber, wherein the chamber further comprises flow disrupter means disposed so as in use to restrict air flowing from the base of the chamber in an axial direction.

Viewed from a second aspect the invention provides a dry powder inhaler incorporating a cyclone chamber comprising at least one air inlet and an outlet wherein the configuration of the air inlet and the shape of the chamber is such that in use a reverse flow cyclone is set up in the chamber, wherein the chamber further comprises flow disrupter means disposed so as in use to restrict air flowing from the central region of the base of the chamber towards the outlet.

Thus it will be seen by those skilled in the art that in accordance with the invention a physical obstruction is provided in the chamber to restrict the main cyclonic vortex flow from reaching the base of the chamber. This in turn prevents medicament powder particles which have not yet been deaggregated from their lactose carrier particles, and which will tend to move towards the base, from being prematurely forced out of the chamber by such generally axial flow (i.e. in a direction generally along or parallel to the axis of the cyclone chamber) or with a substantial axial component of motion. This allows for a further reduction in the chamber size and/or an increase in the dose size accommodated therein without having an adverse impact on the Fine Particle Fraction, i.e. the proportion of particles exiting the mouthpiece which are small enough to be inhaled.

The flow disrupter means could take one of many different forms. In a set of preferred embodiments the flow disrupter means comprises a surface extending across the central axis of the chamber which restricts axial flow and encourages the majority of the air flow to reverse at the flow disrupter rather than extending to the bottom of the chamber. The result of this is that the cyclone structure effectively terminates at the flow disrupter surface. However the Applicant has found that with appropriate choice of the dimensions of the surface, there is a sufficient flow of circulating air to entrain powder in the base of the chamber into the main airflow.

The flow disrupter means in accordance with at least preferred embodiments of the present invention are intentionally configured to restrict the main vortex flows to the region above the flow disrupter surface, as stated. This is in contrast to the embodiment shown in FIG. 7 of WO2006/061637, in which the base of the cyclone chamber generally conforms to part of the surface of a toroid in order to enhance the reverse-cyclone flow pattern, particularly in that region. The raised inner feature at the bottom of that cyclone therefore has sidewalls that generally slope, in order to assist the movement of large particles up them and towards the central axial flow of the cyclone. In the preferred embodiments of the present invention, by contrast, the sidewalls of the flow disrupter member are generally steep-sided (and thus, for example, in some embodiments, at least 40% of the flow disrupter's volume is above its mid-point), in order to prevent the large particles moving in to a closer distance from the chamber axis until they reach or nearly reach the level of the top of the flow disrupter. Although some embodiments may comprise flow disrupter surfaces with non-planar raised tops, in each case powder is forced nearly to the level of the top of the flow disrupter before it can start to move inwardly to the chamber axis.

The Applicant has found that to achieve the greatest benefit in accordance with the invention the surface should be supported such that the support(s) do(es) not extend into the area between the walls of the chamber and the area immediately below the surface, or at least not significantly so. This could be achieved in a number of ways. For example in one set of potential embodiments the surface is supported by one or more struts connected to a wall or walls of the chamber, The strut(s) could be horizontal, vertical, diagonal or a combination thereof. In a subset of such embodiments one or more struts is/are connected to a side wall of an upper part of the chamber. In another subset of such embodiments an axially extending strut supports the surface.

In another set of embodiments the surface is supported from below by one or more protrusions from the base. The protrusion (s) may have parallel or substantially parallel sides, sides which taper downwardly from the surface to the base or any other shape of reduced volume beneath the surface. The protrusion may be a discrete support member for a separate disrupter surface member but, more conveniently, the surface simply comprises an upper face of the protrusion.

In one set of embodiments the disrupter surface is mounted on a rotatable mounting. By allowing the disrupter surface to rotate, frictional losses of energy can be reduced in some circumstances.

When viewed from a third aspect the invention provides a dry powder inhaler incorporating a cyclone chamber having an axial flow disrupter surface extending across the axis of the chamber, said axial flow disrupter surface being provided by or supported by a support that does not extend into the area between the walls of the chamber and the area immediately below the surface.

The flow disrupter surface may extend perpendicularly to said axis.

When viewed from a fourth aspect the invention provides a dry powder inhaler incorporating a cyclone chamber having one or more protrusions from the base of the chamber, wherein at least 40% of the volume enclosed by said protrusion, or by the envelope of said protrusions, is above the mid-point of the height of the protrusion or envelope. This criterion ensures that the protrusion can provide a flow disrupter surface where the protrusion beneath it does not extend out significantly from beneath the surface.

In both the third and fourth aspects of the invention set out above, it is preferred that the chamber has an air inlet and that the configuration of the air inlet and the shape of the chamber are such that in use a reverse flow cyclone is set up in the chamber.

In a preferred set of embodiments the chamber comprises a cylindrical protrusion extending axially up from the base. It will be recognised by those skilled in the art that even a nominally cylindrical protrusion will in practice be at least slightly flared in practice to permit withdrawal of a corresponding moulding tool, e.g. when formed by plastic injection moulding.

The flow disrupter means may comprise a surface extending substantially perpendicularly across the central axis of the cyclone chamber as set out above. In one set of embodiments such a surface is provided by a protrusion. However the flow disrupter means could take other forms. For example the protrusion could be open-ended at its proximal end e.g. forming a hollow tube. Instead of a single protrusion, a plurality of protrusions could be provided e.g. closely spaced to act as a single ‘virtual’ protrusion of similar effective dimensions to the envelope of the protrusions.

In preferred embodiments the flow disrupter surface is continuous, but this is not essential; the surface could be discontinuous e.g. with perforations, slots, apertures or the like defined therein. It could be smooth or textured/patterned.

The protrusion(s) may have a planar or at least substantially planar upper surface. In other embodiments it or they may take other forms e.g. conical or pyramid-shaped (the number of faces of the pyramid not being important) and be convex or concave. Such forms may, in some circumstances, be beneficial in helping to stabilise and locate the cyclone which terminates on the surface.

In preferred embodiments the cyclone chamber comprises a vortex finder. As is known to those skilled in the art, a vortex finder is a projection surrounding the chamber outlet which acts in use to allow exit only of particles circulating up to a maximum radius defined by the dimensions of the vortex finder. In accordance with such embodiments the effective length of the reverse flow cyclone is the axial distance between the bottom of the vortex finder and the top of the flow disrupter.

This axial distance is referred to hereinafter as the ‘vortex core length’. Although the Applicant has found that the vortex core length could be shorter, in preferred embodiments the vortex core length is at least 5 mm. It may for example be between 5 mm and 12 mm, or between 6 mm and 11 mm, or between 7 mm and 10 mm, or between 8 mm and 9 mm, e.g. 8.5 mm. Equally the vortex core length could be between 5 mm and 9 mm or between 6 mm and 9 mm or between 7 mm and 9 mm.

The width of the flow disrupter surface or member can be selected as appropriate to give a balance between being large enough to restrict axial flow below it to a sufficient extent but not too large that there is insufficient helical flow to the base to entrain powder. In a set of preferred embodiments the chamber is provided with one or more pitched air inlet channels to assist in directing swirling air down alongside the projecting flow disrupter member, thereby helping to better entrain powder near the base of the chamber. A further consideration where the flow disrupter member comprises a cylindrical or similar projection from the base is that if the projection is too wide, there will be insufficient space between it and the wall of the chamber to accommodate powder or allow it to be dispersed.

In a set of preferred embodiments the width of the flow disrupter surface or the ‘envelope’ width of the protrusion(s) (hereinafter “disrupter width”) is less than the bore of the vortex finder. More specifically the Applicant has found that there is a relationship between the optimum width of the disrupter surface or protrusion(s), the vortex core length and the bore of the vortex finder. Accordingly in a set of preferred embodiments the disrupter width is less than the vortex finder bore by an amount x times the vortex core length where x is in the range 0.2 to 0.35 preferably 0.25 to 0.31, preferably 0.27 to 0.29, preferably approximately 0.28. Other ratios may, however, be appropriate (e.g. depending on air inlet and outlet configurations, powder loading, etc).

The interior of the cyclone chamber may have any appropriate form which gives the desired air-flow characteristics. In preferred embodiments the cross-sectional area of the chamber decreases in a direction away from the air inlet; this encourages formation of a reverse flow cyclone as is desirable in the preferred embodiments. Preferably such decrease in cross-section continues at least until the flow disrupter or the top of the flow disrupter protrusion(s). Below this, since it is intended that the main vortex structure does not extend below the flow disrupter, the cross-section of the chamber could continue to decrease, but equally it could remain constant or even increase again.

In some embodiments the base of the cyclone chamber is provided with structure for creating eddy currents and thereby enhancing the turbulence of the flow at the base of the chamber. This turbulence may help to entrain powder in the air flow, especially when the flow first starts. The structure may comprise a plurality of protrusions e.g. teeth-like protrusions.

The dimensions of the cyclone chamber may vary dependent upon the application.

In one set of preferred embodiments the overall height of the cyclone chamber (measured from the outlet to the base of the chamber) is less than 20 mm, preferably less than 15 mm. In one sub-set of embodiments the height is between 8 and 12 mm. Where a vortex finder is provided, as is preferred, the outlet is considered to be the lower end of the vortex finder. In some embodiments a bypass airflow is provided to allow a fraction of the air drawn through the device to bypass the cyclone chamber. This can reduce overall inhalation system pressure drop (and hence improve patient comfort and compliance). However a bypass air flow is not essential.

Again the maximum width of the cyclone chamber is dependent on application. In one set of preferred embodiments it is less than 10 mm, preferably less than 8 mm. In one sub-set of embodiments it is between 5 and 7 mm.

Where provided, the width of the flow disrupter surface or envelope of protrusions is similarly dependent on application but in one set of preferred embodiments it is between 0.5 and 3 mm, preferably between 1 and 2 mm. In one sub-set of embodiments it is between 1.25 and 1.75 mm.

Where provided, the height of the protrusion or the maximum or average height of the plurality of protrusions is less than 5 mm, e.g. between 2 mm and 4 mm. In one set of embodiments the height is between 2.5 and 3.5 mm. In another set of embodiments the height is between 0.5 mm and 2.5 mm, e.g. between 1 mm and 2 mm, such shorter protrusions serving to reduce frictional wall losses in circumstances where low cyclone loadings permit their use. For greater powder loadings in a given cyclone, taller flow disrupter protrusions may be required to inhibit the movement of powder to the central axis from where it can be prematurely lost.

The elevation of the flow disrupter surface above the base of the chamber is preferably 20% or more of the height of the chamber, preferably between 20% and 40%, e.g. between 20% and 30% or between 25% and 30%.

Where references are made herein to directions such as up/down, top/bottom etc. there should be understood to be relative to the cyclone chamber having its axis vertical with the air outlet at the top. However such a frame of reference is arbitrary in that the cyclone chamber need not be in such a vertical orientation during use.

The air inlet is preferably arranged to direct air entering the chamber helically around the edge of the chamber to establish a reverse-flow cyclone,. A single inlet or multiple inlets may be provided. As previously mentioned, in a set of preferred embodiments the inlet(s) is/are pitched.

The flow disrupter surface or protrusion may have any shape or cross-section e.g. oval, or any polygon but in preferred embodiments it is circular. In such cases references herein to the width of the surface or protrusion should be read as its diameter.

The cyclone chamber could be provided integrally as part of an inhaler—e.g. as a single chamber intended for re-use or as part of a single-use disposable inhaler. Alternatively it could be provided as a separate package, typically containing the required powdered medicament, for insertion into an inhaler. The package could comprise only a single chamber or a plurality thereof. In one preferred set of embodiments, multiple cyclones with flow disrupter members or surfaces as described above are provided in non-refillable multiple dose inhalers in a ring or multiple rings arrangement or in an array. It will be appreciated that the beneficial features of the invention are provided by the chamber itself, regardless of how this is incorporated into, or used with, an inhaler. Thus when viewed from a further aspect the invention provides a cyclone chamber for a dry powder inhaler comprising at least one air inlet and an outlet wherein the shape of the chamber is such that in use a reverse flow cyclone is set up in the chamber, wherein the chamber further comprises flow disrupter means disposed so as in use to restrict air flowing from the base of the chamber in an axial direction.

When viewed from a yet further aspect the invention provides a cyclone chamber for a dry powder inhaler, said chamber having an axial flow disrupter surface extending across the axis of the chamber, said axial flow disrupter surface being supported by a support that does not extend into the area between the walls of the chamber and the area immediately below the surface.

The flow disrupter surface may extend substantially perpendicularly to said axis.

When viewed from a yet further aspect the invention provides a cyclone chamber for a dry powder inhaler, said chamber having one or more protrusions from the base of the chamber, wherein at least 40% of the volume enclosed by said protrusion, or by the envelope of said protrusions, is above the mid-point of the height of the protrusion or envelope.

All of the optional and preferred features set out herein in relation to the inhaler aspects of the invention are applicable equally to the corresponding cyclone chamber aspects of the invention.

Certain embodiments of the invention will now be described, by way of example only, with reference to the accompanying drawings in which:

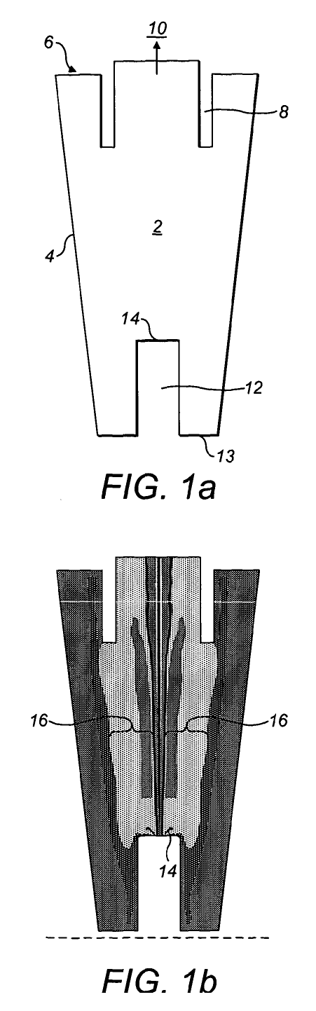

FIG. 1a is simplified view of a cyclone chamber in accordance with an embodiment of the invention;

FIG. 1b is a view similar to FIG. 1a showing air flow in the chamber in use;

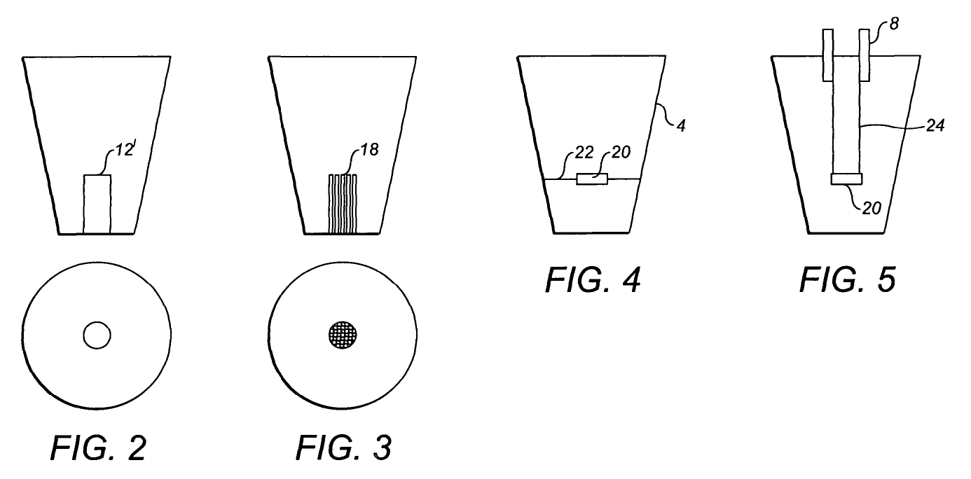

FIGS. 2-23 show various schematic representations of example alternative embodiments of the invention; and

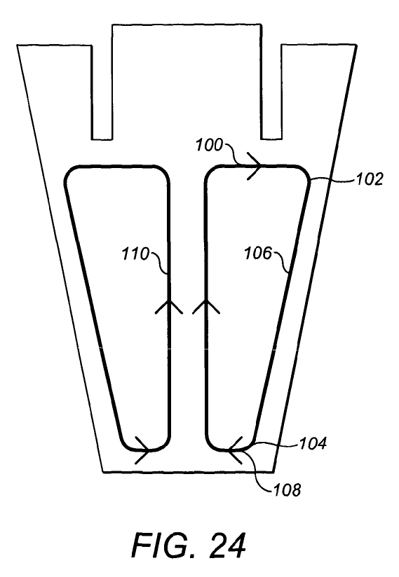

FIG. 24 shows schematically and for the purposes of explanation only, some of the secondary flow motions of large carrier particles within a reverse flow cyclone without a flow disrupter means as described herein.

In order to assist understanding of the operation of embodiments of the invention, an explanation will first be given , with reference to FIG. 24, of the functioning of a reverse flow cyclone chamber not configured in accordance with the invention.

FIG. 24 illustrates schematically one way to visualise some of the powder particle motions that can take place within a reverse flow cyclone chamber. The swirling motion of the free vortex within the cyclone chamber causes air (and particles) to tend to be flung outwardly 100 towards the outer walls of the chamber, causing a quasi-static pressure field wherein the local pseudo-static pressure increases with radius from the central axis of the cyclone, i.e. the local pressure near the top of the cyclone 102 (large radius) exceeds that near the bottom 104 of the cyclone (smaller radius), leading to a pressure gradient down the outer walls of the chamber, down which the larger powder particles (e.g. lactose carrier particles), which are held in the boundary layer of the air flow and which are thus generally less able to follow the swirling cyclonic flow, consequently creep 106. Upon reaching the base of the chamber, these larger particles can then tend to be forced inwards 108 by the local pressure gradient, before joining the generally axial 110 recirculating secondary flow motion, shown in FIG. 24. However, if these particles move in a substantially axial direction away from the central region of the base of the chamber, without sufficient lateral or helical motion to fling them out of the inner forced vortex region before they reach the top of the cyclone chamber, they can to be prematurely lost from the chamber (e.g. with medicament particles not yet deagglomerated from them by sufficient exposure to the cyclonic flows within the chamber). This can lead to an undesired level of emission of non-respirable medicament.

FIG. 1a shows a simplified representation of a cyclone chamber in accordance with the invention for use as part of a dry powder inhaler. The cyclone chamber 2 is generally frusto-conical in shape and so is of circular cross-section with downwardly tapering walls 4. Although not shown, towards the top of the chamber 2 are provided one or more air inlets 6 which are directed approximately tangentially and partially downwardly so that in use air enters the chamber in a helically swirling direction. Also at the top of the chamber is a downwardly protruding annular wall section 8 which surrounds the outlet 10 of the chamber. This annular wall section 8 is known in the art as a vortex finder.

The cyclone chamber of FIG. 1 as thus far described is essentially similar to and operates according to the same principles as those disclosed and discussed in WO 2006/061637. However, this chamber differs in that it has an upwardly vertically extending cylindrical protrusion 12 from the base of the chamber 13 which defines a circular axial flow disrupter surface 14 at the top or distal end thereof. The disrupter surface 14 is thus supported by the cylindrical protrusion 12 extending from the base 13 of the chamber. It will be noticed that the diameter of the disrupter surface 14 and corresponding protrusion 12 is narrower than the bore of the vortex finder 8. In an exemplary arrangement, the diameter of the flow disrupter surface 14 is 1.5 mm and the bore of the vortex finder 8 is 3.18 mm. The difference between these two dimensions is 28% of the distance between the bottom of the vortex finder 8 and the disrupter surface 14, which in this case is 6 mm. This latter dimension is referred to hereinafter as the vortex core length.

In use, the cyclone chamber 2 forms part of a dry powder inhaler. A powdered medicament and lactose carrier particles are provided in the inhaler for enabling a user to inhale the former. The powder could be provided in the cyclone chamber 2 itself or could be provided in a suitable ante-chamber and entrained by the air entering the air inlet 6.

Air is drawn through the mouthpiece (not shown) by a user, so drawing air out of the outlet 10 of the chamber and creating a negative pressure in the chamber which draws air (and possibly powder) in through the air inlet(s) 6. At first air enters the chamber 2 and exits through the outlet 10 without being in a well-defined flow pattern. However, as the flow of air into the chamber begins to build, it begins to circulate around the edge of the chamber in a helical motion. As is described in WO 2006/061637, the tapering cross-section of the chamber encourages a reversal of direction of the cyclonic flow so that a strong, counter-rotating flow travels up the centre axis of the chamber and out of the outlet 10.

The presence of the flow disrupter surface 14 means that for the bulk of the air, the reversal of flow direction takes place at the disrupter surface 14, and not at the base of the chamber 13. This means that powder collected in the bottom part of the chamber around the edge of the protrusion 12 is subjected only to a relatively slow helical cyclonic motion and not to the rapid upward axial motion of the central vortex. The flow disrupter surface 14 can therefore be seen as effectively having forced the main vortex structure to terminate at the surface rather than continuing to the bottom of the chamber. The outcome of this is that there is a significantly reduced tendency for large particles, in which the medicament and lactose have not become deaggregated, to be swept up into the central upward axial airflow and out of the outlet 10. Instead, the large particles reaching the bottom of the chamber are only allowed back into the highly swirling region above the flow disrupter at a significant distance from the axis of the chamber, thereby causing them to acquire substantial lateral and/or helical motion and to be flung back out for further swirling, wall collisions and recirculation. Accordingly, there is a much greater opportunity for all of the powder in the chamber 2 to be properly deaggregated and only particles that are sufficiently small to exit through the vortex finder 8 as is described in WO 2006/061637. The air flow is illustrated in FIG. 1b which shows a shaded plot obtained from a computational fluid dynamics model of the chamber. In general, the paler shades of grey 16 represent the regions of greatest swirl velocity, showing how the central vortex core of rapidly moving air exists only above the height of the flow disrupter surface 14 and not below it.

As will have been seen, the presence of the flow disrupter restricts the inwards movement of particles at the base of the cyclone under the influence of the local pressure gradient. In particular, it inhibits the particles moving all the way to the central region of the base of the cyclone chamber. They cannot, therefore, join the generally axial secondary flow motion close to the central axis of the chamber, but instead are kept away from the central axis. Once they reach the top of the flow disrupter they enter the highly swirling region that only exists above the top of the flow disrupter, but they are sufficiently far off-centre (by a minimum distance related to the radius of the flow disrupter means) to acquire substantial lateral/helical motion and are therefore much more likely to be flung outwards and recirculated rather than to prematurely escape from the cyclone chamber via the outlet with the main airflow.

The arrangement described above allows the overall size of the cyclone chamber 2 to be reduced as compared to an arrangement without the protrusion 12 and flow disrupter surface 14, but without sacrificing a high Fine Particle Fraction (FPF)—i.e. a high proportion of particles exiting the chamber are sufficiently small (typically 5 microns or less) to be inhaled into the deep lung.

The remaining Figures depict schematically various examples of possible alternative embodiments which can achieve a similar effect. The embodiment in FIG. 2 is very similar to that shown in FIGS. 1a and 1b (the air inlet and vortex finder are not shown for simplicity), the only difference here being that rather than the chamber being moulded with a hollow re-entrant portion to form the protrusion 12, here the protrusion 12′ is fixed into the base of a frusto-conical chamber.

FIG. 3 shows an embodiment whereby the single protrusion shown in the previous embodiments is replaced by a plurality of narrow and closely spaced protrusions 18 within a circular-cylindrical envelope. The envelope of protrusions has the same dimensions as the solid protrusion and operates in exactly the same way.

FIGS. 4 and 5 show how the flow disrupter surface can be provided by a disc member 20 but rather than being provided by or supported by a protrusion from the base of the chamber, the disc is supported by horizontal or vertical struts 22, 24 from the side wall 4 or the vortex finder 8 respectively, e.g. by three horizontal struts 22. These embodiments demonstrate that it is not essential for the disrupter surface to be supported from the base of the chamber. Although these embodiments are likely to be more expensive and/or difficult to manufacture, they have the advantage of maximising the volume of the chamber beneath the disrupter surface 20 which thus maximises the amount of powder which can be accommodated there. Indeed, such arrangements may permit an even greater reduction in the overall size of the chamber.

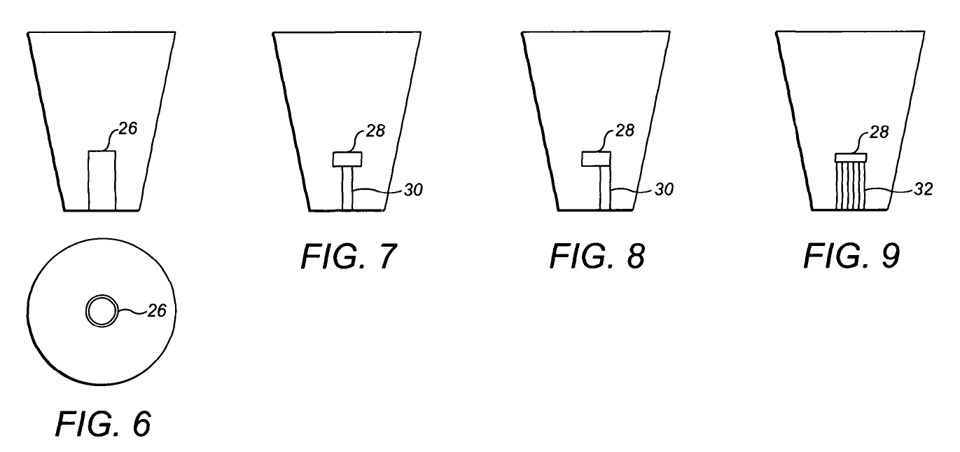

FIG. 6 illustrates an embodiment with a circular-section, open-ended tubular protrusion 26 from the base of the chamber. This can be seen particularly from the plan view of the chamber in the lower part of FIG. 6. In this embodiment, the protrusion 26 acts as a flow disrupter.

FIG. 7 illustrates an arrangement where the flow disrupter surface 28 is supported from the base of the chamber by a support member 30 which is narrower than the width of the flow disrupter surface 28. This illustrates that a cylindrical protrusion is not essential. This embodiment may have the benefit of accommodating a greater volume of powder beneath the flow disrupter surface 28.

FIG. 8 illustrates a variant of the embodiment of FIG. 7 in which the support member 30 is not on the central axis of the chamber. More generally, the support for the flow disrupter surface can take any convenient shape or form although in preferred embodiments it does not extend significantly laterally outside the vertical projection or footprint of the flow disrupter surface.

FIG. 9 illustrates an embodiment in which the flow disrupter surface 28 is supported by a plurality of supports 32 connected to the base.

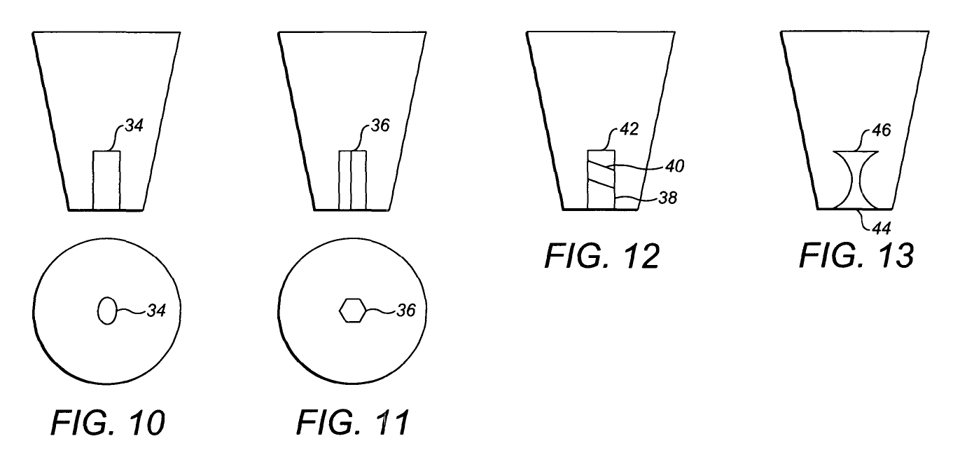

FIGS. 10 and 11 illustrate embodiments in which respective protrusions 34, 36 from the base are provided but where the cross-sectional shape of the protrusion is non-circular. In FIG. 10, the cross-sectional shape is oval and in FIG. 11, it is hexagonal. Of course, any other suitable regular or irregular shape could be employed.

In the embodiment of FIG. 12, a generally cylindrical protrusion 38 is provided which has helical contours 40 on its surface which may assist the desired powder-entraining helical airflow in the region of the chamber below the flow disrupter surface 42 at the top of the protrusion.

FIG. 13 illustrates another example of a protrusion 44 providing a flow disrupter surface on its distal face 46, where the protrusion is not cylindrical. Another such example is illustrated in FIG. 17. In this case the protrusion 48 is frusto-conical.

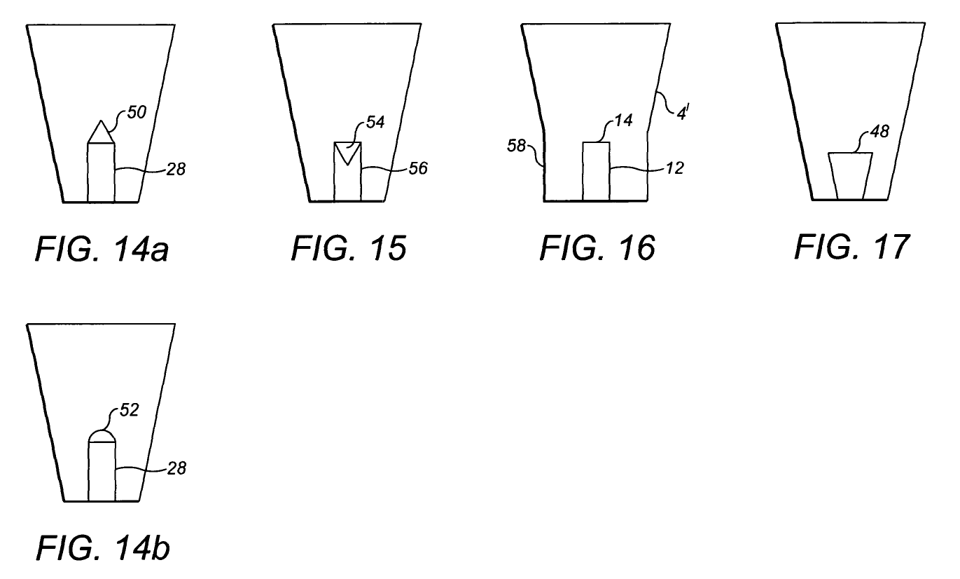

FIG. 14 shows two examples of a flow disrupter surface which is non-planar. In FIG. 14a the flow disrupter surface 50 is conical and in FIG. 14b, the flow disrupter surface 52 is dome-shaped. In both cases, however, the support member 28 is cylindrical and remains within the vertical projection of the respective flow disrupter surface 50, 52.

FIG. 15 shows an embodiment in which the flow disrupter surface 54 at the top of the protrusion 56 is concave (e.g. as a concave conical surface or as a concave pyramidal surface) rather than convex as in FIGS. 14a and 14b or flat as in the other embodiments. FIG. 16 illustrates an embodiment in which the side wall of the chamber 4′ is not uniformly tapering but has a straight section 58 which has a similar vertical extent to the protrusion. As explained previously, there is no reverse flow cyclone structure beneath the flow disrupter surface 14 at the top of the protrusion 12.

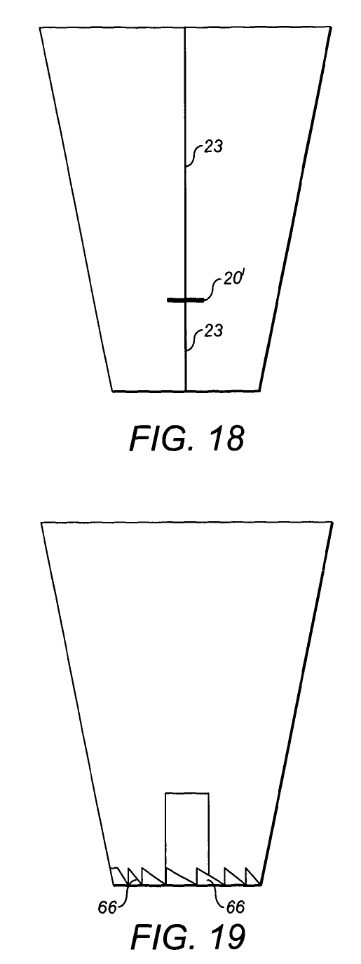

FIG. 18 shows an embodiment in which a flow disrupter surface in the form of a thin disk 20′ is supported by a fine axial strut or wire 23 that runs from the bottom of the cyclone chamber to the centre of the air outlet. In one variation of this type of embodiment, the disk 20′ is free to rotate on wire 23 as the vortex flow develops, thereby reducing frictional losses of energy in the system.

FIG. 19 shows an embodiment in which the flow disrupter member is surrounded by a ring of teeth-like features 66 at the bottom of the cyclone which create eddies and encourage greater turbulence in the air flow in the powder holding region below the flow disrupter surface. This turbulence serves to help to entrain the powder in the air flow, in particular as the flow first starts.

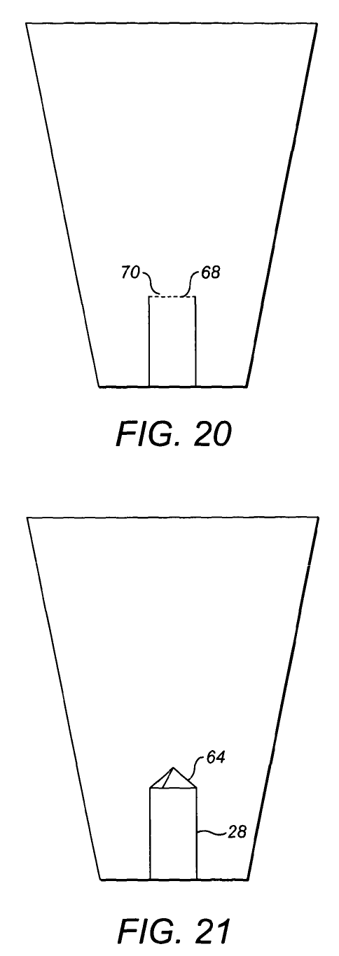

FIG. 20 shows an embodiment in which the flow disrupter surface 68 comprises perforations 70.

FIG. 21 shows an embodiment in which the flow disrupter member comprises a cylindrical support 28 and a pyramidal top 64.

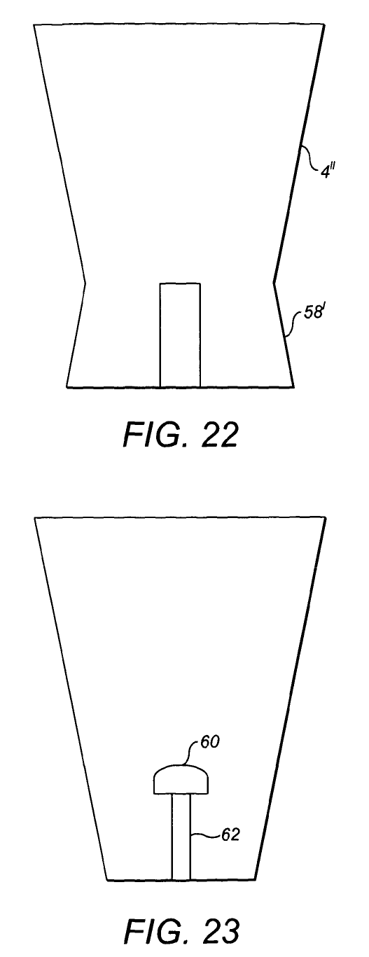

FIG. 22 shows an embodiment in which the side wall of the chamber 4″ is not uniformly tapering but has a bottom section 58′ that flares outwardly towards the bottom of the chamber and which has a similar vertical extent to the protrusion.

Finally, FIG. 23 shows an embodiment in which the flow disrupter surface 60 is dome-shaped and of larger outer diameter than the diameter of its support 62.

It will be appreciated by those skilled in the art that the embodiments shown above are merely a small number of examples of the many ways in which a flow disrupter member or flow disrupter surface can be employed in accordance with the invention. There will be many other ways of providing such a surface or member within a cyclone chamber which have the same effect and follow the same principles as those described above.

1. A dry powder inhaler incorporating a cyclone chamber comprising at least one air inlet and an outlet wherein the configuration of the air inlet and the shape of the chamber is such that in use a reverse flow cyclone is set up in the chamber, wherein the chamber further comprises a flow disrupter disposed so as in use to restrict air flowing from the base of the chamber in an axial direction.

2. A dry powder inhaler incorporating a cyclone chamber comprising at least one air inlet and an outlet wherein the configuration of the air inlet and the shape of the chamber is such that in use a reverse flow cyclone is set up in the chamber, wherein the chamber further comprises a flow disrupter disposed so as in use to restrict air flowing from the central region of the base of the chamber towards the outlet.

3. An inhaler as claimed in claim 1 wherein said flow disrupter comprises a surface extending across the central axis of the chamber which restricts axial flow and encourages the majority of the air flow to reverse at the flow disrupter rather than extending to the bottom of the chamber.

4. An inhaler as claimed in claim 1 wherein said flow disrupter comprises a surface supported such that the support(s) substantially do(es) not extend into the area between the walls of the chamber and the area immediately below the surface.

5. An inhaler as claimed in claim 1 wherein said flow disrupter comprises a surface supported by one or more struts connected to a wall or walls of the chamber.

6. (canceled)

7. (canceled)

8. An inhaler as claimed in claim 1 wherein said flow disrupter comprises a surface supported from below by one or more protrusions from the base.

9. An inhaler as claimed in claim 8 wherein the surface comprises an upper face of the protrusion(s).

10.-14. (canceled)

15. An inhaler as claimed in claim 1 wherein the chamber comprises a cylindrical protrusion extending axially up from the base.

16. An inhaler as claimed in claim 1 wherein said flow disrupter comprises a surface extending substantially perpendicularly across the central axis of the cyclone chamber.

17. An inhaler as claimed in claim 16 wherein said surface is provided by a protrusion.

18. An inhaler as claimed in claim 1 wherein the cyclone chamber comprises a vortex finder.

19. (canceled)

20. An inhaler as claimed in claim 18 wherein the disrupter width is less than the bore of the vortex finder.

21. (canceled)

22. An inhaler as claimed in claim 1 wherein the cross-sectional area of the chamber decreases in a direction away from the air inlet.

23. An inhaler as claimed in claim 22 wherein said decrease in cross-section continues at least until the flow disrupter or the top of the flow disrupter protrusion(s).

24. An inhaler as claimed claim 1 wherein the base of the cyclone chamber is provided with structure for creating eddy currents and thereby enhancing the turbulence of the flow at the base of the chamber.

25. An inhaler as claimed in claim 24 wherein said structure comprises a plurality of protrusions.

26. An inhaler as claimed in claim 1 wherein the overall height of the cyclone chamber is less than 20 mm.

27. An inhaler as claimed in claim 1 comprising a bypass airflow to allow a fraction of the air drawn through the inhaler to bypass the cyclone chamber.

28.-30. (canceled)

31. An inhaler as claimed in claim 1 comprising a flow disrupter surface whose elevation above the base of the chamber is 20% or more of the height of the chamber.

32. An inhaler as claimed in claim 1 comprising an air inlet arranged to direct air entering the chamber helically around the edge of the chamber to establish a reverse-flow cyclone.

33. An inhaler as claimed in claim 1 wherein the chamber is provided with one or more pitched air inlet channels.

34. (canceled)

35. A cyclone chamber for a dry powder inhaler comprising at least one air inlet and an outlet wherein the shape of the chamber is such that in use a reverse flow cyclone is set up in the chamber, wherein the chamber further comprises a flow disrupter disposed so as in use to restrict air flowing from the base of the chamber in an axial direction.

36.-38. (canceled)

39. A package for insertion into an inhaler comprising one or more cyclone chambers as claimed in claim 35.

40. A cyclone chamber for a dry powder inhaler comprising at least one air inlet and an outlet wherein the shape of the chamber is such that in use a reverse flow cyclone is set up in the chamber, wherein the chamber further comprises a flow disrupter disposed so as in use to restrict air flowing from the central region of the base of the chamber towards the outlet.
